(19) United States
(12) Patent Application Publication
(10) Pub. No.: US 2013/0012791 A1
(43) Pub. Date: Jan. 10, 2013

(54) Portable Device Holder

(76) Inventor: Jason M. Coles, Burlington, NJ (US)

(21) Appl. No.: 13/542,615

(22) Filed: Jul. 5, 2012

Related U.S. Application Data
(60) Provisional application No. 61/505,737, filed on Jul. 8, 2011.

(51) Int. Cl. A45F 5/00; A61B 5/021; A61B 5/01; A61B 5/0205

(52) U.S. Cl. 600/301; 294/137; 294/167; 600/500; 600/549

(57) ABSTRACT

A portable device holder apparatus and system is provided where the portable device holder comprises a device gripper and a glove unit to hold and support a portable device.

### CROSS-REFERENCE TO RELATED PATENT APPLICATION

This application claims the benefit under 35 U.S.C. §119(e) of U.S. Provisional Patent Application. No. 61/505,737, filed on Jul. 8, 2011 in the U.S. Patent and Trademark Office, the entire disclosure of which is hereby incorporated by reference.

### FIELD OF THE INVENTION

The present application relates to a portable device holder. Particularly, the present invention provides for an apparatus and system for holding and/or supporting a portable device.

### BACKGROUND OF THE INVENTION

Conventional device holders do not provide a comfortable, secure grip of a device with flexibility because they do not provide for a secure grip of the device holder. Conventional device holders that have a strap for placing a hand do not comfortably secure the device holder to the hand. In such case, the only way to tightly secure a hand against the device holder would be to adjust a strap (such as making the strap tighter, which in turn puts pressure around the hand, thus potentially restricting blood flow and cutting off circulation for the person holding the device.

Embodiments of the present invention address at least these drawbacks associated with the conventional art, and provide further advantages and features over the conventional art.

### SUMMARY OF THE INVENTION

As noted above, exemplary embodiments of the present invention address at least the above problems and/or disadvantages, and provide at least the advantages described below.

Exemplary embodiments of the present invention relate to a novel portable device holder apparatus and/or system, for holding and/or supporting a portable device (such as a portable electronic device) with a human hand.

An exemplary embodiment of the present invention provides for a portable device holder apparatus for holding and supporting a portable device, the holder comprising a human glove for encasing a human hand, the glove comprising a palm side for attaching to a device gripper, and a device gripper for encasing a portable device, the device gripper comprising a back side and a front side, wherein the palm side of the glove is coupled to the back side of the device gripper.

An exemplary embodiment of the present invention provides for a portable device holder apparatus for holding and supporting a portable device, the holder comprising a glove for encasing a human hand and a device gripper for encasing a portable device, and the holder further comprising a rotation unit for rotating and/or swiveling the device gripper unit, wherein the rotation unit is coupled between the glove and the device gripper.

While the present invention has been shown and described with reference to certain exemplary embodiments thereof, it will be understood by those skilled in the art that various changes in form and details may be made therein without departing from the spirit and scope of the present invention as defined by the appended claims.

### BRIEF DESCRIPTION OF THE DRAWINGS

The above and other exemplary features, aspects and advantages of the present invention will become more apparent from the following detailed description of certain exemplary embodiments thereof when taken in conjunction with the accompanying drawings in which:

FIG. 1 exemplifies a front view of the portable device holder showing a device gripper unit, according to an exemplary embodiment of the present invention.

FIG. 2 exemplifies a back view of the portable device holder showing a glove unit, according to an exemplary embodiment of the present invention.

FIG. 3 exemplifies the top view of the portable device holder showing a glove unit and a device gripper unit, according to an exemplary embodiment of the present invention.

FIG. 4 exemplifies a side of the portable device holder including a rotation unit, according to an exemplary embodiment of the present invention.

FIG. 5 exemplifies a profile view of the portable device holder showing a flexible and/or hinged gripper unit for allowing a portable device to be tilted while still being partially and/or fully encased in the gripper, according to an exemplary embodiment of the present invention.

FIG. 6 exemplifies a portable device holder showing one or more sensor(s) in the glove unit, according to an exemplary embodiment of the present invention.

FIG. 7 exemplifies a side of the portable device holder including a wired connection from the portable device to the sensor(s) on the glove wherein the sensor(s) can make contact with a human hand to detect a pulse or other vital signs, according to an exemplary embodiment of the present invention.

FIG. 8 exemplifies a side of the portable device holder including a glove with at least one sensor that makes contact with a human hand to detect a pulse or any other vital sign from the human being, a wire from the sensor(s) to a receiver unit comprised in the glove, and a wired or wireless connection from the receiver unit to the portable device, according to an exemplary embodiment of the present invention.

Throughout the drawings, like reference numerals will be understood to refer to like elements, features and structures.

### DETAILED DESCRIPTION OF EXEMPLARY EMBODIMENTS

The matters exemplified in this description are provided to assist in a comprehensive understanding of exemplary embodiments of the present invention described with reference to the accompanying drawing figures. Accordingly, those of ordinary skill in the art will recognize that various changes and modifications of the exemplary embodiments described herein can be made without departing from the scope and spirit of the present invention. Also, descriptions of well-known functions and constructions are omitted for clarity and conciseness. Likewise, certain naming conventions, labels and terms as used in the context of the present disclosure are, as would be understood by skilled artisans, non-limiting and provided only for illustrative purposes to facilitate understanding of certain exemplary implementations of the embodiments of the present invention.

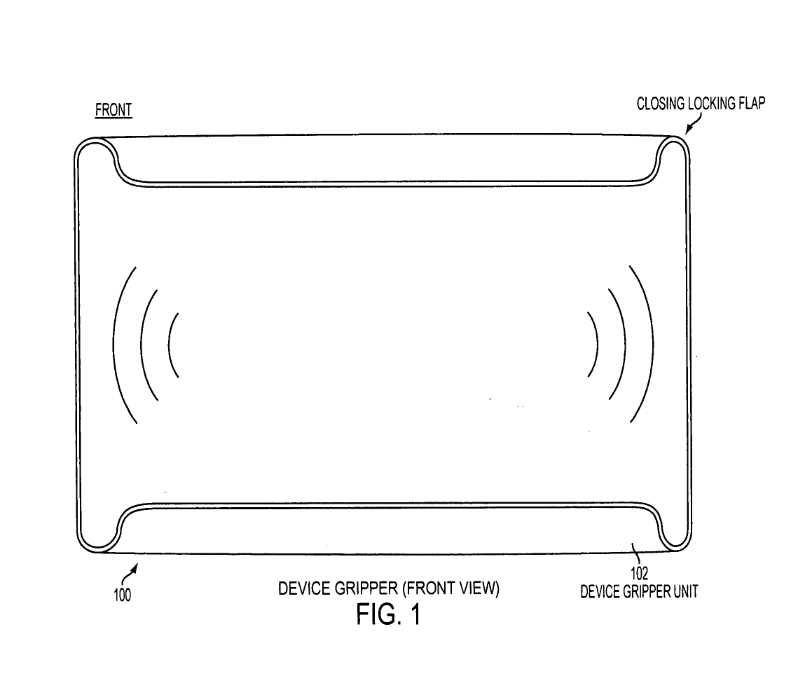

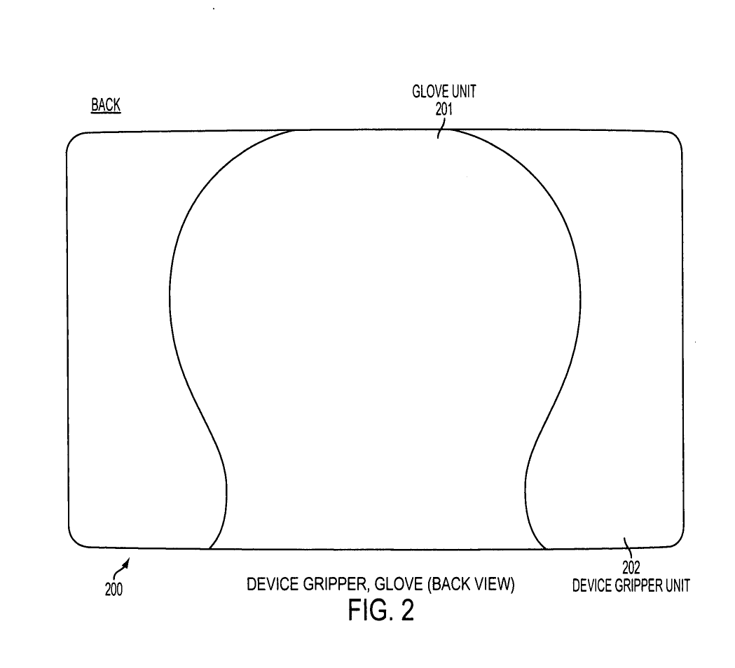

FIGS. 1-7 exemplify the portable device holder 100, 200, 300, 400, 500, 700 according to an exemplary embodiment of the present invention, that allows a person to use the portable device holder to hold and support a portable device (such as an electronic device, a computer, or a tablet computer (for example, an iPad made by Apple, Inc.)) without the person having to directly grip the portable device (i.e., an electronic device) with the fingers on their hand, while the portable device holder provides a firm and secure hold/grasp/grip of the device.

FIGS. 1-5 and 7 exemplify a portable device holder 100, 200, 300, 400, 500, 700 according to exemplary embodiments of the present invention. For example, the portable device holder 100, 200, 300, 400, 500, 700 comprises a device gripper unit 102, 202, 302, 402, 502, 702 for securely gripping a device (such as an electronic device), and a glove unit 201, 301, 401, 501, 701 where a person's hand can slide inside (like a glove or a mitten).

According to an exemplary embodiment of the present invention, the device gripper of the portable device holder grips an electronic device such that a human interface for the electronic device appears in an open space on the front side of the device gripper allowing the human holder/user of the electronic device to interface with the electronic device while the electronic device is secure in the device gripper unit 102, 402, 702.

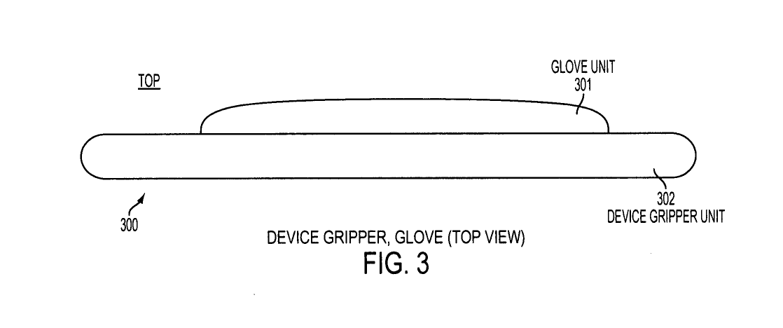

According to an exemplary embodiment of the present invention, as shown in FIG. 3, the glove unit 301 is coupled directly to the device gripper unit 302. The glove unit is coupled to the device gripper unit by applying an adhesive in between the glove unit and device gripper unit, stitching the glove unit to the device gripper unit, fastening the glove unit to the device gripper unit with a button and/or pin (metal, plastic or synthetic material), and/or melting the glove unit to the device gripper unit, or any combination thereof.

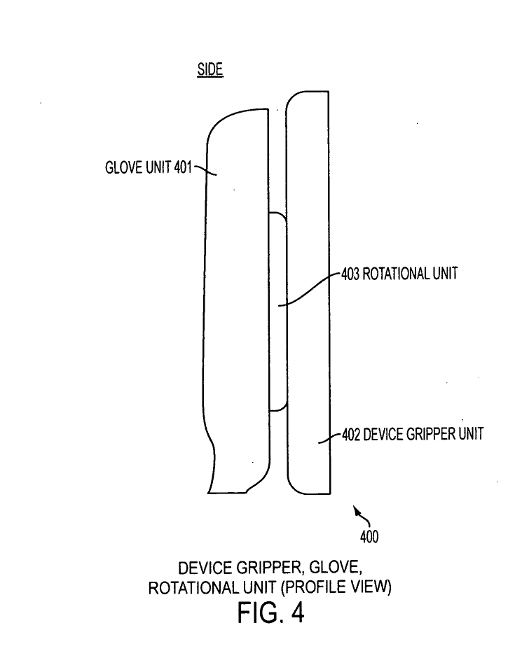

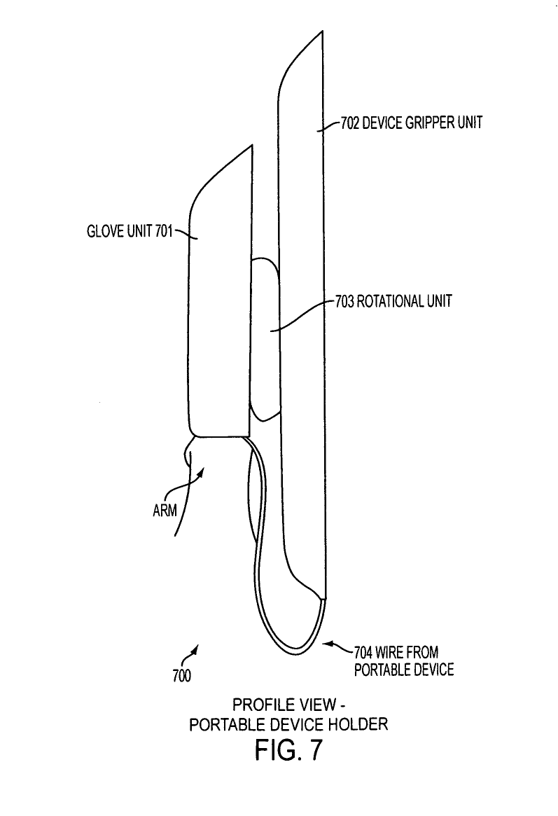

According to an exemplary embodiment of the present invention, as shown in FIGS. 4 and 7, the portable device holder 400 comprises a rotational unit 403, 703 coupled to the device gripper unit 402, 702 and the glove unit 401, 701, and attached between the device gripper 402, 702 and the glove 401, 701. The rotational unit 403 is comprised of a hard plastic or metal material, or a combination thereof. The rotational unit 403 is coupled to the device gripper unit 402 by applying an adhesive in between the rotational unit and the device gripper unit and/or by fastening the rotational unit to the device gripper unit with a button and/or pin (metal, plastic or synthetic material), or any combination thereof. Further, The rotational unit 403 is coupled to the glove unit 401 by applying an adhesive in between the rotational unit and the glove unit and/or by fastening the rotational unit to the glove unit with a button and/or pin (metal, plastic or synthetic material), or any combination thereof.

According to an exemplary embodiment of the present invention, as shown in FIGS. 4 and 7, the device gripper unit 402 can be rotated and/or pivoted/swiveled in relation to the glove unit 401 by pivoting and/or swiveling at the rotational unit 403. The device gripper unit 402 can be rotated 360 degrees (in a partial or full circle) in relation to the glove unit 401, providing the benefit of allowing the person to adjust the rotational angle of the face of the device to adjust the view.

For example, the person holding an electronic device 504 with the portable device holder 400, 700 of the present invention (like holding a tray) can rotate (or spin around) the device gripper unit 100, 402, 702 to show the device 504 to someone that is facing them such as to re-orient the display on the device 504. The device gripper unit 402, 702 can be rotated by approximately 180 degrees while the person holding the device with the portable device holder 400, 700 has their hand in the glove unit 401, 701, and then the device gripper unit 402, 702 can be rotated again in either direction.

For example, according to an exemplary embodiment of the present invention, the device gripper unit 402, 702 can be securely rotated bi-directionally in a 360-degree direction while the portable device gripped by the device gripper unit 402, 702 faces the person holding the portable device holder with the glove unit 401, 701. The person holding the portable device using the portable device holder 400, 700 of the present invention can rotate the device (gripped by the device gripper unit 402, 702) in order to orient the device to be in a portrait or landscape view, or any view in between.

For example, a person holding an electronic device with the portable device holder can rotate, spin, and/or swivel the electronic device to present a different view to the person facing the person holding the portable device holder.

Exemplary embodiments of the present invention provide the added benefit for allowing the person holding the device with the glove unit 401, 701 to pivot/swivel the device in order to change the angle or tilt of view of the portable device in relation to the glove unit 401, 701, in any direction. For example, this can be helpful in order to adjust to a glare or make it easier to display to another person.

According to an exemplary embodiment of the present invention, the portable device holder 400, 700 comprises a rotational unit 403 that can be configured to allow the device gripper unit 402, 702 to be partially or fully dis-mounted or separated from the glove unit 401, 701. For example, this provides the benefit of allowing the device gripper unit 402, 702 to be placed on a docking station (for landscape or portrait display setting).

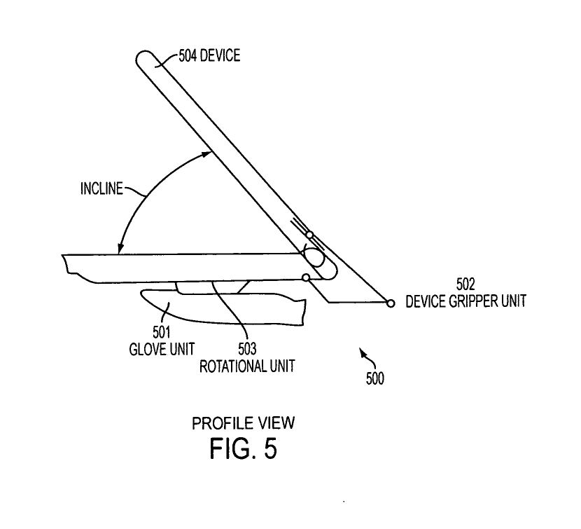

According to an exemplary embodiment of the present invention, FIG. 5 exemplifies a profile view of the portable device holder showing a flexible and/or hinged gripper for allowing a portable device to be tilted while still being encased in the gripper unit 502. This provides the benefit of allowing the person using the device to tilt just the portable device for a more convenient view (e.g., to avoid glare), without having to tilt the glove unit 501 and rotational unit 503. Further, according to an exemplary embodiment of the present invention, the portable device holder 500 can be placed on a surface (such as a table) with the glove unit 501 on the surface, such that the portable device 504 is facing up, and the device 504 can be tilted to a resting inclined position, as shown in FIG. 5, where the device 504 is still encased and supported by one side of the device gripper unit 502. According to this exemplary embodiment, at least one of the sides of the device gripper unit 502 that is encasing the device 504 comprises a flexible material or is hinged in order to allow the device 504 to be tilted. For example, when a user of the device 504 places the portable device holder 500 on a table with the glove unit 501 at the bottom, the user can apply downward pressure on the flexible/hinged side of the device gripper unit 502 and/or simultaneously applying upward pressure on the opposite side of the device 504 such that the opposite side of the device 504 lifts up while the flexible/hinged side of the device gripper unit 502 presses downward, while the device gripper unit 502 is still encasing the device.

According to an exemplary embodiment of the present invention, as shown in FIGS. 1-5 and 7, the device gripper unit 102, 202, 302, 402, 502, 702 is made of a hard plastic material and/or any material that is able to securely hold/grip/grasp a device. For example, the device gripper unit can be comprised one or more material including but not limited to vinyl, plastic, leather, leather, and any other material capable to hold a device in place, or any combination thereof. The device gripper unit 102, 202, 302, 402, 502, 702 can hold/grip/grasp a device such that a device can be gripped/held in place securely and the device does not fall out of the device gripper, while allowing the removal of the device if extra pressure is applied to remove the device from the device gripper.

According to an exemplary embodiment of the present invention, as shown in FIGS. 1-7, the glove unit 201, 301, 401, 501, 601, 701 is made of a soft material that is comfortable for a human hand. For example, the glove unit 201, 301, 401, 501, 601, 701 can be made of a soft cotton fiber on the inside portion of the glove that contacts the hand and a nylon material on the outside of the glove, or any combination thereof.

According to an exemplary embodiment of the present invention, the glove unit 201, 301, 401, 501, 701 comprises the shape of an n-finger glove (for example, a 5-finger glove), a mitten, a pouch, a strap and/or a pocket, or any combination thereof that allows a human hand to be inserted into the glove unit.

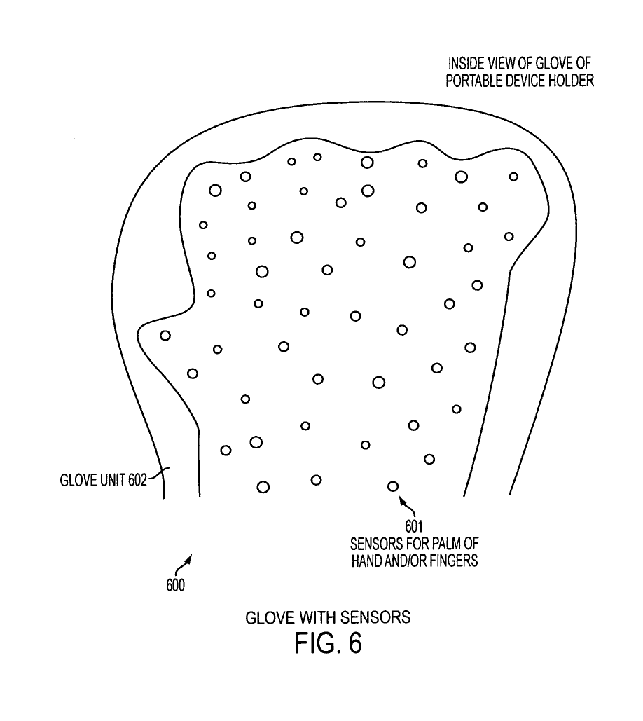

According to an exemplary embodiment of the present invention, as shown in FIG. 6, the portable device holder 600 comprises one or more sensor(s) 601 on the glove unit 602, where the sensor(s) can sense a pulse, temperature and/or vital signs of the person holding the portable device gripper. For example, the sensor(s) 601 can be placed on the inside and/or outside the glove unit 602 for determining the person's heart rate (similar to a ECG, EKG reading). Further, the sensor 601 can transmit the sensed pulse, temperature and/or vital sign reading to the portable device. For example, the transmitted reading can be provided to a software application executed on the portable device that calculates the person's pulse rate or any other statistic based on the transmitted reading.

According to exemplary embodiments of the present invention, the sensor(s)601 transmit via wired and/or wireless electronic transmission, one or more electronic signals of the sensed information based on the reading. For example, as shown in FIGS. 6 and 7, an electronic signal can be transmitted from sensor(s) 601 on/in the glove unit 602, 701 to the portable device 600 via a wire 704 that connects the sensor(s) 601 with the portable device.

FIG. 7 exemplifies the side view of the portable device holder 700 including a wired connection 704 from the portable device (encased in the device gripper unit 702, not shown) to the sensor(s) 601 on the glove 701 where the sensors 601 can make contact with a human hand to detect a pulse or other vital signs, according to an exemplary embodiment of the present invention.

Further, according to exemplary embodiments of the present invention, the device gripper unit 102, 202, 302, 402, 502, 702 comprises holes and/or spaces for interfacing with jacks, plugs, wires, etc. The device gripper unit 102, 202, 302, 402, 502, 702 comprises a hole for allowing a user to plug in a power cord, headphones, audio/video cables, and/or any other type of interface requiring a physical connection with the device (designed based on the type, make, and model of the device).

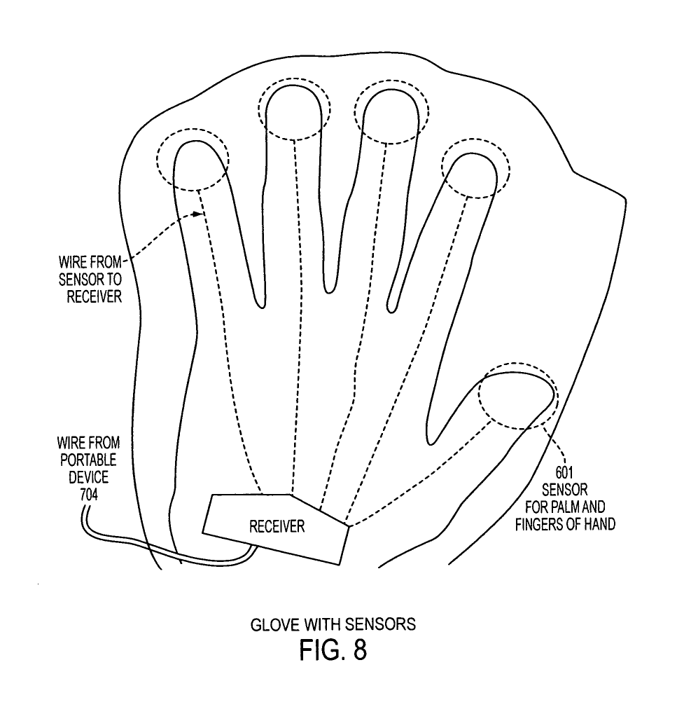

According to an exemplary embodiment of the present invention, as shown in FIG. 8, the portable device holder includes a glove and at least one sensor 601, attached thereto, that makes contact with a human hand (for example, along the fingers or the palm of the hand) that detects a pulse or any other vital sign from the human being. The glove further comprises a wire from each sensor to a receiver unit comprised in the glove. The receiver unit receives signals from the sensor(s), and transmits the signals, via a wired or wireless connection 704, to the portable device (for example, the wireless connection can be implemented via a Bluetooth connection). According to an exemplary embodiment of the present invention, the receiver unit processes (for example, consolidating, compressing, or altering) the received signal prior to transmission to the portable device. Alternatively, the receiver unit transmits the received signals without further processing the signal.

As shown in FIG. 8, the sensors can be attached inside the glove such that the sensors make a stable contact with at least some portion of a human hand inserted in the glove.

Although exemplary embodiments of the present invention have been disclosed for illustrative purposes, those skilled in the art will appreciate that various modifications, additions, and substitutions are possible, without departing from the scope of the present invention. Therefore, the present invention is not limited to the above-described embodiments, but is defined by the following claims, along with their full scope of equivalents.

What is claimed is:

1. A portable device holder apparatus for holding and supporting a portable device, the holder comprising:
a human glove for encasing a human hand, the glove comprising a palm side; and
a device gripper for encasing a portable device, the device gripper comprising a back-side and a front side,
wherein the glove unit is coupled to the back side of the device gripper unit.

2. The apparatus of claim 1, further comprising a rotation unit for rotating and/or swiveling the device gripper unit, wherein the rotation unit is coupled between the glove unit and the device gripper.

3. The apparatus of claim 1, wherein the front side of the device gripper unit comprises space for viewing a portable device.

4. The apparatus of claim 1, wherein the glove unit is directly attached to the device gripper unit.

5. The apparatus of claim 1, wherein the portable device comprises an electronic device.

6. The apparatus of claim 1, wherein the device gripper is detachably coupled to the glove unit.

7. The apparatus of claim 1, wherein the palm side of the glove is coupled to the back-side of the device gripper.

8. The apparatus of claim 1, wherein the glove comprises at least one sensor, wherein the sensor senses a pulse, temperature and/or vital signs of the person holding the portable device.

9. The apparatus of claim 1, wherein the device gripper comprises a first side that is flexible and/or hinged for allowing a portable device to be tilted while still being encased in the gripper unit.

10. A portable device holder for holding and supporting a portable device, the holder comprising:
a pouch configured to receive a human hand; and
a device gripper configured to encase a portable electronic device,
wherein the pouch is coupled to the device gripper.

11. The apparatus of claim 8, wherein the glove comprises a receiver to receive a signal from the at least one sensor, and to transmit a signal based on the signal from the sensor to the portable device.
